(19) United States
(12) Patent Application Publication
(10) Pub. No.: US 2020/0367750 A1
(43) Pub. Date: Nov. 26, 2020

(54) PHOTOACOUSTIC SYSTEM FOR ACCURATE LOCALIZATION OF LASER ABLATION CATHETER TIP POSITION AND TEMPERATURE MONITORING DURING ABLATION PROCEDURES

(71) Applicants: HENRY FORD HEALTH SYSTEM, Detroit, MI (US); WAYNE STATE UNIVERSITY, Detroit, MI (US)

(72) Inventors: Mohammad MEHRMOHAMMADI, Farmington Hills, MI (US); Loay KABBANI, Bloomfield Hills, MI (US); Nicole A. KENNEDY, Livonia, MI (US); Yan YAN, Detroit, MI (US)

(73) Assignees: HENRY FORD HEALTH SYSTEM, Detroit, MI (US); WAYNE STATE UNIVERSITY, Detroit, MI (US)

(21) Appl. No.: 16/966,321

(22) PCT Filed: Feb. 1, 2019
(86) PCT No.: PCT/US2019/016269

§ 371 Date: Jul. 30, 2020

Related U.S. Application Data
(60) Provisional application No. 62/625,121, filed on Feb. 1, 2018.

(51) Int. Cl. A61B 5/00; A61B 18/24

(52) U.S. Cl. CPC A61B 5/0036; A61B 2018/00577; A61B 18/24; A61B 5/0095

(57) ABSTRACT

A system for monitoring an ablation procedure in a target tissue includes a first light source for delivering light to the target tissue to generate photoacoustic signals and a second light source for delivering light to the target tissue for ablation therapy. The system further includes a beam mixer for receiving light from the first and second light sources to create a combined light beam. An ablation catheter including a single optical fiber receives the combined light beam from the beam mixer, wherein the combined light beam is emitted from a tip of the ablation catheter to perform simultaneous ablation therapy and photoacoustic monitoring of the ablation procedure in the target tissue in real time.

### CROSS-REFERENCE TO RELATED APPLICATIONS

This application claims the benefit of U.S. provisional application Ser. No. 62/625,121 filed Feb. 1, 2018, the disclosure of which is hereby incorporated in its entirety by reference herein.

### TECHNICAL FIELD

Embodiments relate to a photoacoustic system for monitoring a laser ablation procedure in a target tissue, such as monitoring catheter tip position and temperature.

### BACKGROUND

Chronic venous insufficiency (CVI) is caused by the reflux in one-way valves of the lower extremity venous system. CVI causes edema, discoloration, dermatitis, and ulceration in the affected limbs. Endovascular ablation of the superficial veins is a safe, minimally invasive, and efficacious treatment for moderate to severe CVI of the superficial veins of the lower extremity. This procedure has a success rate of 90-95%.

Endovenous laser therapy (EVLT) or endovenous laser ablation (EVLA) is a minimally-invasive method that is used to ablate superficial varicose veins. After accessing the desired vein, a thin fiber is inserted through a sheath into the diseased vein. Laser light is emitted through the fiber, and as the fiber is pulled back through the vein it delivers light energy to the surrounding tissue. The targeted tissue reacts with the light energy, generating heat and causing the vein to seal shut. Complications may occur from this therapy in the form of heat-induced thrombosis extending into the deep veins. There is also concern for recanalization of treated veins.

Current methods of ultrasound-guided endovenous ablation using laser-based fibers often lack precision in identifying the true location of the ablation fiber tip. Additionally, existing systems lack the ability to non-invasively determine the tissue temperature at the activated fiber tip in real time.

### SUMMARY

In one or more embodiments, a system for monitoring an ablation procedure in a target tissue includes a first light source for delivering light to the target tissue to generate photoacoustic signals and a second light source for delivering light to the target tissue for ablation therapy. The system further includes a beam mixer for receiving light from the first and second light sources to create a combined light beam. An ablation catheter including a single optical fiber receives the combined light beam from the beam mixer, wherein the combined light beam is emitted from a tip of the ablation catheter to perform simultaneous ablation therapy and photoacoustic monitoring of the ablation procedure in the target tissue in real time.

In one or more embodiments, a system for monitoring an ablation procedure in a target tissue includes a pulsed laser for delivering light pulses to the target tissue to generate photoacoustic signals and a continuous wave laser for delivering continuous wave light energy to the target tissue for ablation therapy. The system further includes a beam mixer for receiving light from the pulsed laser and the continuous wave laser to create a combined light beam. An ablation catheter including a single optical fiber receives the combined light beam from the beam mixer, wherein the combined light beam is emitted from a tip of the ablation catheter to simultaneously perform ablation therapy and to generate photoacoustic signals in the target tissue. The system further includes an ultrasound transducer for detecting the generated photoacoustic signals, and a processor in communication with the ultrasound transducer for processing the photoacoustic signals to create photoacoustic images of the tip of the ablation catheter to allow tracking of the tip in real time simultaneous to ablation therapy.

In one or more embodiments, a system for monitoring an ablation procedure in a target tissue includes a pulsed laser for delivering light pulses to the target tissue to generate photoacoustic signals and a continuous wave laser for delivering continuous wave light energy to the target tissue for ablation therapy. The system further includes a beam mixer for receiving light from the pulsed laser and the continuous wave laser to create a combined light beam. An ablation catheter including a single optical fiber receives the combined light beam from the beam mixer, wherein the combined light beam is emitted from a tip of the ablation catheter to simultaneously perform ablation therapy and to generate photoacoustic signals in the target tissue. The system further includes an ultrasound transducer for detecting the generated photoacoustic signals, and a processor in communication with the ultrasound transducer for processing the photoacoustic signals to monitor a temperature at the tip of the ablation catheter in real time simultaneous to ablation therapy.

### BRIEF DESCRIPTION OF THE DRAWINGS

FIG. 1 is a perspective view of a system combining a laser ablation source and a photoacoustic imaging laser source using a single fiber according to an embodiment;

FIG. 2 is a side view of the system according to an embodiment;

FIG. 3 is a schematic illustration of the system according to an embodiment;

FIG. 4 is a cross-sectional image of a vessel-mimicking phantom with combined ultrasound and photoacoustic images of the laser fiber, where ultrasound (US) shows the whole fiber body and photoacoustic (PA) imaging only indicates the location of the fiber tip;

FIG. 5A is a schematic illustration of an experimental setup for fiber tip tracking where a vessel-mimicking phantom with two vessels, one straight and one angled, is made of tissue-mimicking material and the fiber is placed in the vessels for image testing;

FIG. 5B is a volumetric ultrasound image of the phantom of FIG. 5A;

FIG. 6 depicts experimental results using ultrasound and photoacoustic imaging according to the disclosed embodiments for the straight fiber of FIGS. 5A and 5B;

FIG. 7 depicts experimental results using ultrasound and photoacoustic imaging according to the disclosed embodiments for the angled fiber of FIGS. 5A and 5B;

FIG. 8 is a schematic illustration of an experimental setup for fiber tip tracking in a simulated blood vessel filled with human blood, where an ultrasound probe was moved in a linear fashion from distal to proximal ends of the catheter to acquire cross-sectional images;

FIG. 9 depicts experimental results for the fiber tip tracking of FIG. 8, where ultrasound and photoacoustic images according to the disclosed embodiments of the catheter cross-sections before, at, and after the tip are demonstrated;

FIG. 10 is a schematic illustration of an experimental setup for ultrasound and photoacoustic fiber tip tracking of a straight fiber in a porcine tissue sample according to the disclosed embodiments;

FIG. 11 depicts experimental results for the fiber tip tracking of FIG. 10, with photoacoustic images shown in the top row and ultrasound images shown in the bottom row;

FIG. 12 is a schematic illustration of an experimental setup for ultrasound and photoacoustic fiber tip tracking of an angled fiber in a porcine tissue sample according to the disclosed embodiments;

FIG. 13 depicts experimental results for the fiber tip tracking of FIG. 12, with photoacoustic images shown in the top row and ultrasound images shown in the bottom row;

FIG. 14 is a schematic illustration of an experimental setup for real-time temperature monitoring using ultrasound and photoacoustic imaging according to the disclosed embodiments to evaluate the changes in the amplitude of the photoacoustic signal with changes in the surrounding temperature; and

FIG. 15 depicts experimental results for the temperature monitoring of FIG. 14, showing a graph and photoacoustic imaging of a fiber tip located in different surrounding temperatures ranging from 23 to 85 degrees.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Photoacoustic imaging is an imaging technology that allows for the ultrasonic detection of objects upon radiation of electromagnetic (EM) waves as an excitation. Using light as an excitation source, photoacoustic imaging uses a short, low-power, laser pulse to excite the target tissue. Consequent to rapid but small thermal expansion of the excited tissue, acoustic waves will be generated which convey information about optical properties of the tissue. In addition, these waves only arise from the optically excited region. Photoacoustic signals are also known to convey information about the temperature of the surrounding tissue.

The system disclosed herein uses photoacoustic imaging for monitoring a laser ablation procedure, such that accurate positioning of the ablation fiber (catheter) tip location as well as thermal regulation of delivered energy may be determined more precisely. Simultaneous to laser ablation therapy, the disclosed system provides real-time, accurate fiber tip tracking as well as monitoring of the temperature at the tip of the fiber and thermal dose deposition inside the ablated vein or other tissue. The disclosed system provides images of the tip of a laser ablation catheter, free of known ultrasound artifacts/noise (such as angular dependency and comet tail), and accurate and localized temperature information without the need for light delivery that is external or separate from the ablation catheter for photoacoustic imaging. Specifically, the system uses the same optical fiber to simultaneously carry both high-power continuous wave (CW) laser energy for ablation and low-power laser pulses for generating a photoacoustic signal at the tip of the catheter. The two laser outputs are combined for delivery into a single fiber, such as by using an optical component as described below.

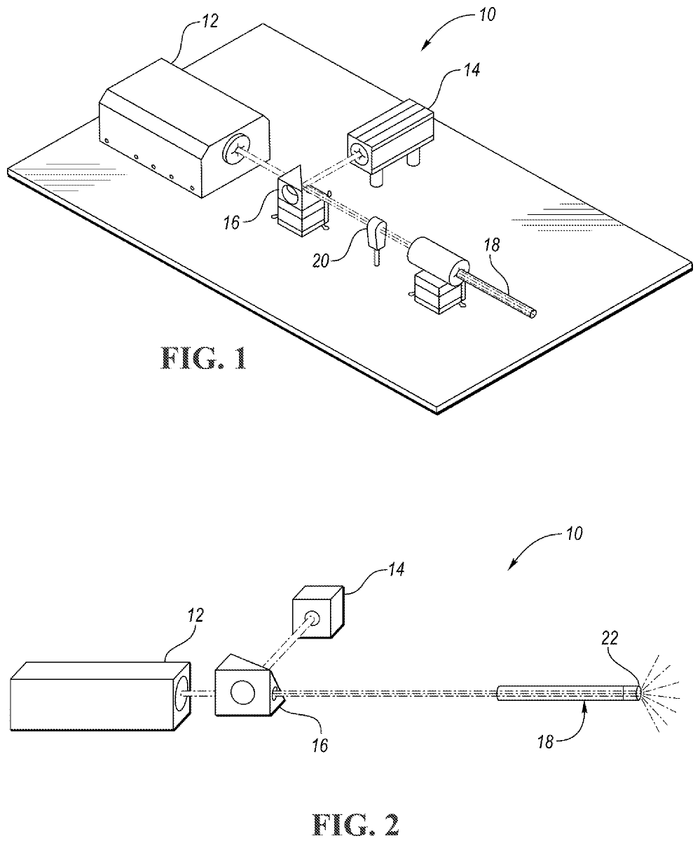

FIGS. 1-3 illustrate an embodiment of the system 10 which combines an imaging laser or first light source 12 for delivering light to a target tissue for generating photoacoustic signals for imaging and a therapy laser or second light source 14 for delivering light to the target tissue for ablation therapy. In the embodiment shown, a pulsed laser 12 for imaging (“Pulsed Laser” or “Laser 1”) is combined with a continuous wave laser 14 (“CW Laser” or “Laser 2”) for ablation therapy using an optical component or beam mixer 16. The pulsed laser 12 may provide pulses with a very short duration, for example, on the order of nanoseconds. In one or more embodiments, the lasers 12, 14 are positioned within the system 10 to deliver light to the beam mixer 16 at different incidence angles, such as positioned at approximately 90 degrees relative to one another as shown in FIGS. 1-3. The ablation therapy wavelengths may be different from and may be spaced apart from the photoacoustic imaging wavelengths (for example, but not limited to, ablation at 1470 nm and photoacoustic imaging at 532 nm). In one embodiment, the beam mixer 16 may be transparent to light from one of the lasers 12, 14 and reflect light from the other laser 12, 14. As a result, light beams from both lasers 12, 14 can be combined and coupled into a single fiber (ablation catheter) 18. It is also contemplated that a single laser that can operate in both continuous wave and pulsed modes could alternatively be used to perform both ablation therapy and photoacoustic imaging in system 10.

In one embodiment, the beam mixer 16 may be a dichroic mirror or block which has different reflection or transmission properties of light at two different wavelengths. In other embodiments, the beam mixer 16 could include a cold mirror reflecting visible light wavelengths while transmitting infrared light wavelengths or could include a hot mirror reflecting infrared light wavelengths while transmitting visible light wavelengths. The beam mixer 16 can be positioned within the system 10 at an incidence angle, typically between 0 and 45 degrees, appropriate to receive, transmit, and reflect light from the lasers 12, 14 as desired. A focal lens 20 may also be positioned within the system 10 to focus light from the beam mixer 16 to the fiber 18.

In the ablation catheter 18, laser light is only emitted from a tip 22 portion of the catheter 18 (FIG. 2). Accordingly, in the target tissue, only a small region near the catheter tip 22 is exposed to laser pulses from the first light source 12, generating photoacoustic signals just at the tip 22 and not along the entire fiber 18. This arrangement allows for selective visualization of only the fiber tip 22 instead of the whole catheter 18 using an ultrasound transducer 24, thus improving the precision of determining the location of the laser fiber tip 22 using system 10. It may also help ensure that a physician or other medical professional does not lose the catheter tip 22 while imaging during the ablation procedure, especially in scenarios where the catheter 18 is bent, twisted or turned out of the ultrasound imaging plane. The omni-directionality of the generated photoacoustic signal makes the photoacoustic image of the tip 22 free of certain ultrasound artifacts, such as angular dependency. In other words, the photoacoustic imaging of system 10 can see the catheter tip 22 independent from the relative angle of the ultrasound transducer 24 and the fiber 18, as long as the imaging plane includes the tip 22.

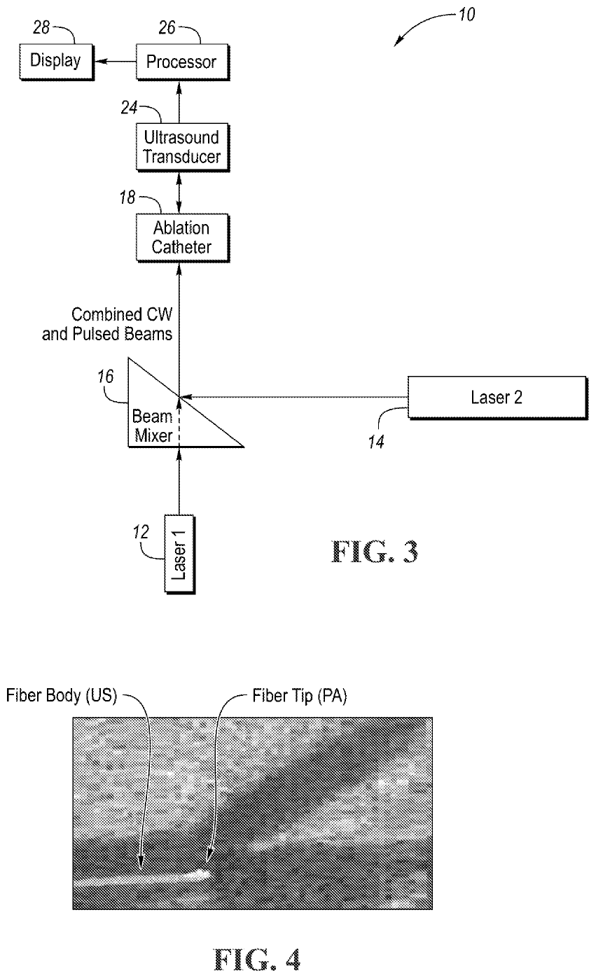

With reference to FIG. 3, the photoacoustic signals received by the ultrasound transducer 24 (receive/echo mode) can be processed by a processor 26 to generate photoacoustic images which are displayed on a display 28, where the processor 26 and display 28 may both be embodied in a computer, for example. Since the photoacoustic signal is only generated at the region close to the fiber tip 22, the generated photoacoustic images are very high contrast (no or minimal background) which increases the contrast-to-noise ratio required for accurate detection of the fiber tip 22. The ultrasound transducer 24 may also transmit ultrasound signals (pulse/echo mode) to the target tissue for generating ultrasound images, representing the anatomical picture of the target tissue. The disclosed system 10 provides more accurate, noise/artifact-free photoacoustic images of the ablation catheter tip 22, which can be superimposed on or co-registered with a gray-scale (or combined gray-scale/Color Doppler) ultrasound background image. As an example, FIG. 4 is a cross-sectional image of a vessel-mimicking phantom with a combined ultrasound image and photoacoustic image of the laser fiber 18, where the ultrasound image shows the whole fiber (catheter) body 18 and photoacoustic image only indicates the location of the fiber (catheter) tip 22, as only the fiber tip 22 generates a photoacoustic signal. With the use of interleaved or sequenced ultrasound and photoacoustic images, the physician can see either an ultrasound image, a photoacoustic image, or both images (combined or overlaid).

System 10 also provides the capability for accurate and localized temperature measurement without the need for using laser energy or any other device that is external to the body or separate from the ablation catheter 18. Temperature monitoring requires calibration, where the calibration process includes testing the system in human blood and determining a relationship (e.g. “lookup table”) between the photoacoustic signal and the temperature, as the strength (amplitude) of the generated photoacoustic signal at the catheter tip 22 will depend on the surrounding temperature. The photoacoustic signal also depends on several other parameters, including light fluence, absorption coefficient of the tissue, and the speed of sound. In contrast to methods which use external illumination for photoacoustic imaging, the light source (from pulsed laser 12) is internal to the disclosed system 10. Therefore, the thermal measurements of system 10 will be independent of fluence and the optical properties of the surrounding tissue since the catheter 18 is either in blood or certain unchanged tissue. In addition, temperature measurements will be highly localized at the catheter tip 22 for temperature/heat deposition monitoring and will not be affected by the tissue path. As such, the disclosed system 10 improves regulation of thermal energy delivery at the activated fiber tip 22 to ensure adequate ablation at all points of the treated vessel or other target tissue. In addition to temperature monitoring, the photoacoustic signals can be used to indicate the thermal damage to the selected vessels or other target tissue (i.e. indicating whether the vessel is thermally ablated or not). This is based on the changes of optical properties of ablated tissue versus non-ablated tissue.

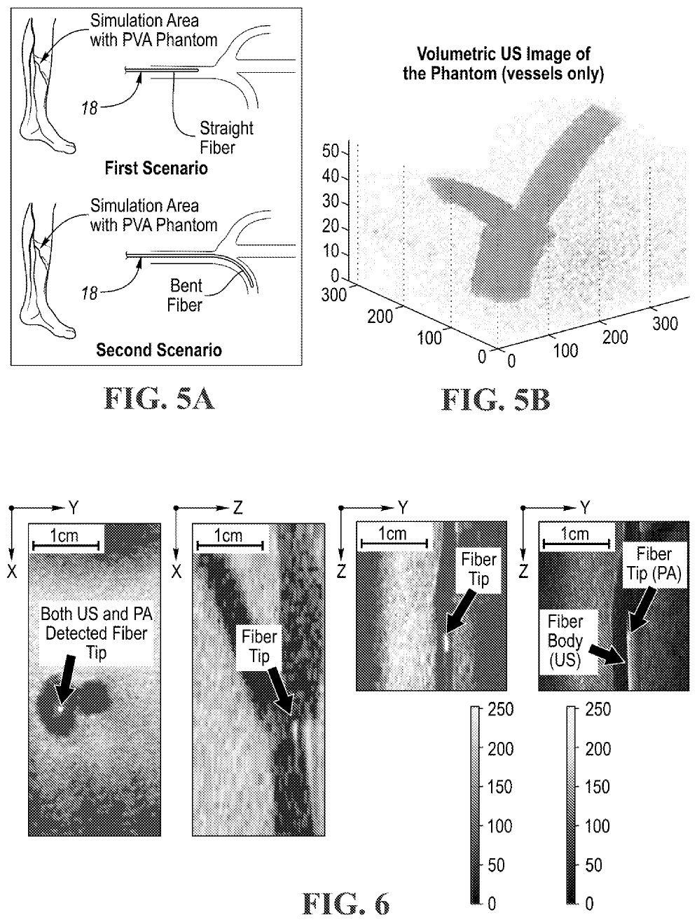

A prototype system was developed, and ultrasound and photoacoustic experiments were conducted in realistic tissue-mimicking phantoms in which one straight vessel and one vessel going out of the imaging plane was made in the material. FIG. 5A is a schematic illustration of an experimental setup for fiber tip tracking for both straight and angled (˜45 degrees) fibers in a polyvinyl alcohol (PVA) phantom. In this experiment, an L7-4 transducer (ATL-Philips) was used with a bandwidth of 4-7 MHz, and a fiber core diameter of 1000 μm was employed. A laser wavelength of 532 nm and fiber tip energy of ˜100 μJ/pulse were used, with a fiber bending angle of 0 degrees (straight) and a scanning resolution of 1 mm. A volumetric ultrasound image of the phantom (vessels only) is shown in FIG. 5B.

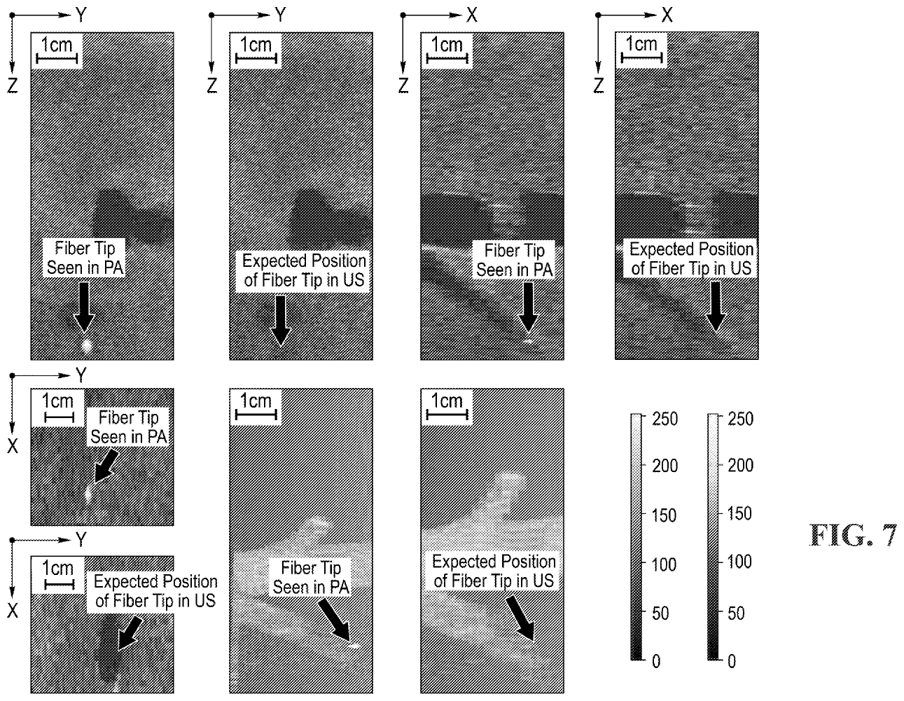

Experimental results for the straight and angled fiber configurations of FIG. 5A are depicted in FIGS. 6 and 7, respectively, where ultrasound and photoacoustic images of the phantom were acquired and rendered into a 3D volume. For easier visualization, after rendering the volumetric images, slices in the transverse, sagittal and coronal planes are demonstrated. As shown in these comparisons of ultrasound and photoacoustic images, while ultrasound loses (cannot visualize) the fiber tip, photoacoustic imaging can easily visualize the tip, even when the fiber is not perpendicular to the ultrasound beam.

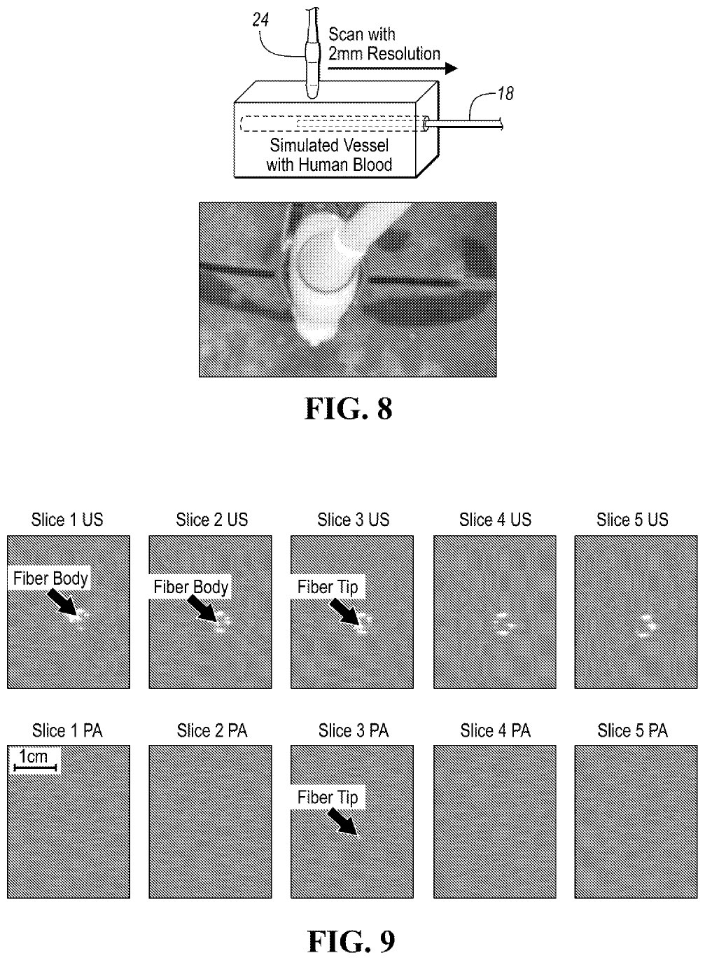

In another experiment, a lower power pulsed laser was tested in human blood. The experimental setup is schematically illustrated in FIG. 8, where an L11-4V transducer (Verasonics) was used with a center frequency of 9.5 MHz. The ultrasound image was created with ray-line beam forming and a frame rate of 5 fps (can also be reconstructed with high frame rate plane wave ultrasound imaging, not shown), while the photoacoustic frame rate was 10 fps. These frame rates are merely exemplary, as higher frame rates could also be used. The ultrasound and photoacoustic results are shown in FIG. 9, where the ultrasound images show the fiber body and tip, while the photoacoustic images only show the tip.

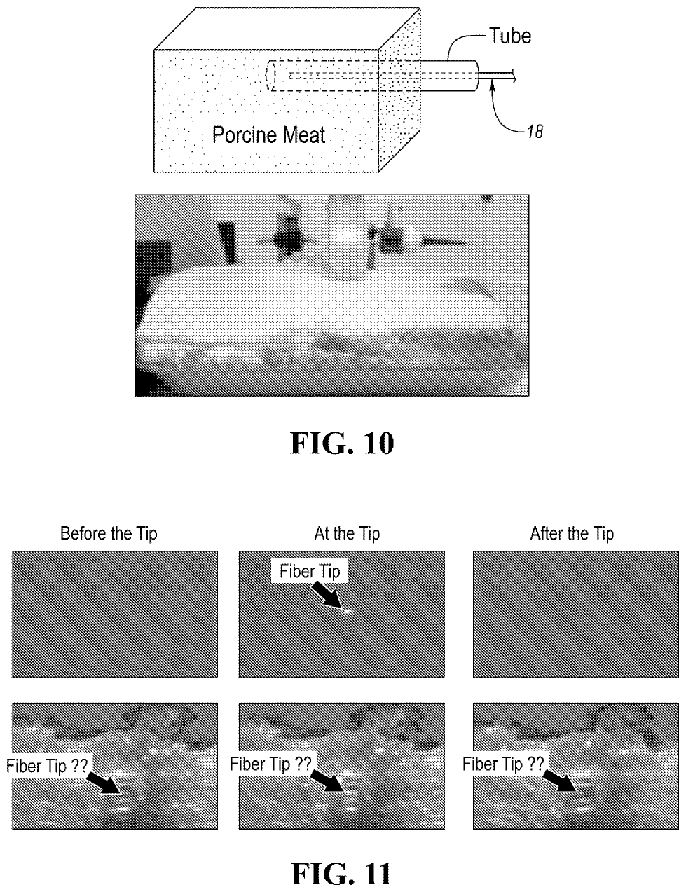

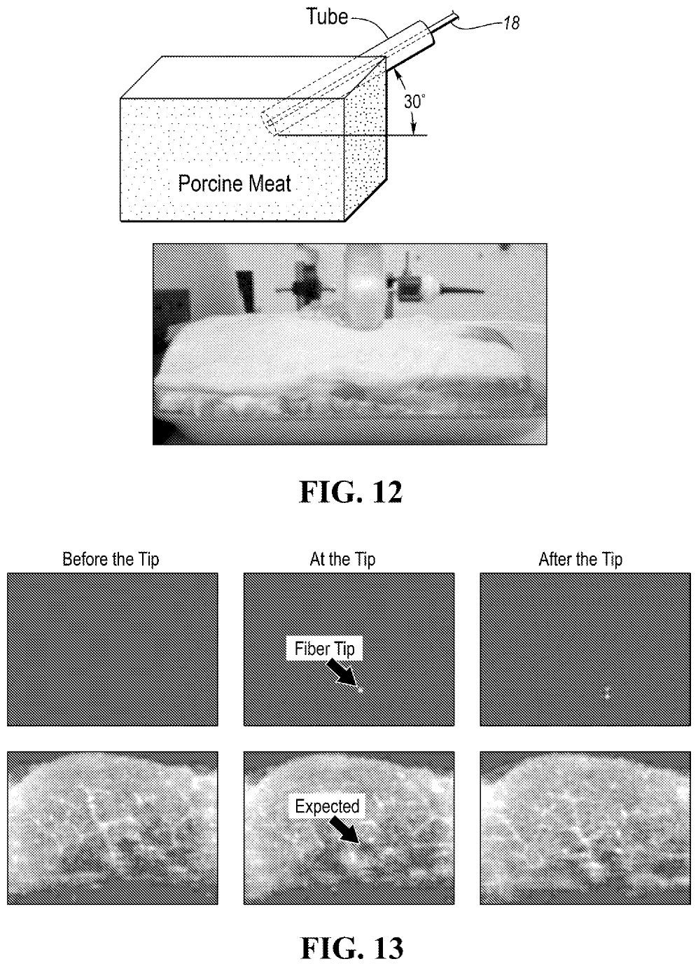

Similar to the tissue-mimicking phantom experiments, fiber tip tracking experiments were also conducted in porcine tissue samples. FIG. 10 is a schematic illustration of fiber tip tracking for a straight fiber in a porcine tissue sample. An L11-4 transducer (Verasonics) was used with a center frequency of 9.5 MHz. A laser wavelength of 532 nm and fiber tip energy of 2000 were used, with a scanning distance of 20 mm, scanning resolution of 1 mm, and an image depth of 40 mm. Of course, it is understood that these values used for all of the studies described herein are not intended to be limiting and that other parameter values could alternatively be used. Experimental results for the configuration of FIG. 10 are depicted in FIG. 11 with photoacoustic images in the top row and ultrasound images in the bottom row. As shown, artifact in the tissue makes tracking the tip with ultrasound imaging difficult. FIG. 12 is a schematic illustration of fiber tip tracking for a fiber bending angle of 30 degrees, with the rest of the parameters the same as those described above for FIG. 10. Experimental results for the configuration of FIG. 12 are shown in FIG. 13 with photoacoustic images in the top row and ultrasound images in the bottom row. Again, it is not possible to visualize the fiber tip with ultrasound alone, but the photoacoustic images detect the tip position accurately.

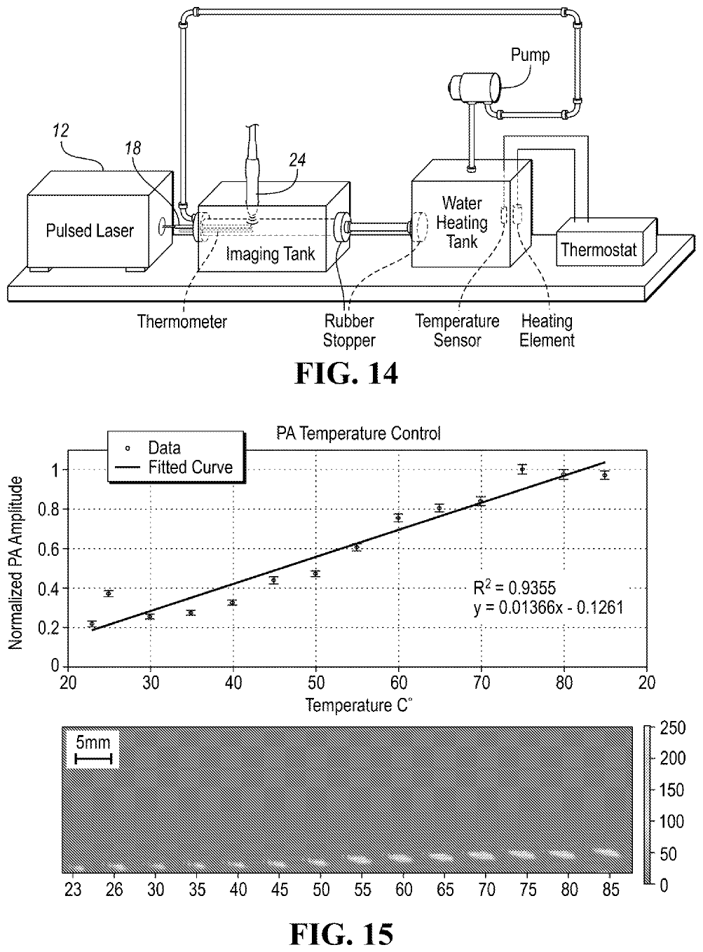

Lastly, the feasibility of using photoacoustic imaging according to the disclosed system to monitor a temperature increase in tissue was tested in a tissue-mimicking phantom. FIG. 14 is a schematic illustration of an experimental setup for performing real-time temperature monitoring using photoacoustic imaging. Water was heated to selected temperatures in a water heating tank by a heating element and measured by a temperature sensor. A pump transferred the heated water to an imaging tank in which the fiber 18, ultrasound transducer 24, and a thermometer were disposed. FIG. 15 illustrates the resulting photoacoustic imaging of the fiber tip located in different surrounding temperatures ranging from 23 to 85 degrees (bottom panel), where variation of the photoacoustic signal is demonstrated by increasing the temperature as shown in the graph (top panel). Therefore, using the disclosed system, the temperature at the tip of the fiber can be measured very accurately and in real time, and thus very localized measurements of the heat deposition within the tissue can be obtained.

There is no existing technology to help accurately locate the tip of the laser ablation catheter within vessels, especially in cases where ultrasound imaging has difficulties visualizing the fiber. Photoacoustic imaging offers significant advantages in visualizing the fiber tip, such as not being affected by the angular dependency in ultrasound and also being completely independent from ultrasound appearance of the tissue. As such, the photoacoustic image of the fiber tip is a background-free image that indicates the location of the tip only. By using photoacoustic imaging simultaneous with laser ablation therapy in a single fiber as in the disclosed system, the fiber tip can be accurately tracked and thermal dose deposition at the tip accurately measured. These advantages can significantly improve clinical endogenous laser ablation procedures at a low cost without requiring a change to existing clinical procedures.

In one example, using photoacoustic imaging to locate the tip of the fiber will simplify catheter tip location at the saphenofemoral junction. This may decrease the incidence of heat-induced thrombosis as well as the time needed to perform the procedure. Measuring the temperature at the catheter tip will help the physician to deliver the desired amount of thermal energy to the target tissue. This thermal energy may be adjusted to accommodate different target vessels as desired.

The photoacoustic system disclosed herein can also be used in a number of applications including, but not limited to, fiber tip detection in breast-conserving surgery (lumpectomy), guided-biopsy procedures, catheter-based photothermal treatment, catheter-based atrial fibrillation ablation, and other applications in which visualization and/or monitoring temperature of an external catheter, fiber, or the like is required.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system for monitoring an ablation procedure in a target tissue, the system comprising:
a first light source for delivering light to the target tissue to generate photoacoustic signals;
a second light source for delivering light to the target tissue for ablation therapy;
a beam mixer for receiving light from the first and second light sources to create a combined light beam; and
an ablation catheter including a single optical fiber which receives the combined light beam from the beam mixer, wherein the combined light beam is emitted from a tip of the ablation catheter to perform simultaneous ablation therapy and photoacoustic monitoring of the ablation procedure in the target tissue in real time.

2. The system of claim 1, wherein the second light source includes a continuous wave laser for creating continuous wave light energy for ablation.

3. The system of claim 1, wherein the first light source includes a pulsed laser for creating light pulses at the tip of the ablation catheter.

4. The system of claim 1, wherein a wavelength of light from the first light source is different than a wavelength of light from the second light source.

5. The system of claim 1, wherein the beam mixer includes a dichroic mirror.

6. The system of claim 1, wherein the beam mixer includes a hot mirror or a cold mirror.

7. The system of claim 1, further comprising a focal lens positioned between the beam mixer and the optical fiber to focus the combined light beam.

8. The system of claim 1, further comprising an ultrasound transducer for detecting the generated photoacoustic signals.

9. The system of claim 8, further comprising a processor in communication with the ultrasound transducer for processing the photoacoustic signals to create photoacoustic images of the tip of the ablation catheter to allow tracking of the tip in real time simultaneous to ablation therapy.

10. The system of claim 9, wherein the photoacoustic images of the tip of the ablation catheter are independent of a relative angle between the ultrasound transducer and the ablation catheter if an imaging plane of the ultrasound transducer includes the tip.

11. The system of claim 9, wherein the ultrasound transducer is configured to transmit ultrasound signals to the target tissue for generating ultrasound images of the target tissue.

12. The system of claim 11, further comprising a display in communication with the processor for displaying the photoacoustic images, the ultrasound images, or both.

13. The system of claim 11, wherein the processor combines or superimposes photoacoustic images of the tip of the ablation catheter with ultrasound images of the target tissue.

14. The system of claim 9, wherein the processor processes the photoacoustic signals to monitor a temperature at the tip of the ablation catheter in real time simultaneous to ablation therapy.

15. A system for monitoring an ablation procedure in a target tissue, the system comprising:
a pulsed laser for delivering light pulses to the target tissue to generate photoacoustic signals;
a continuous wave laser for delivering continuous wave light energy to the target tissue for ablation therapy;
a beam mixer for receiving light from the pulsed laser and the continuous wave laser to create a combined light beam;
an ablation catheter including a single optical fiber which receives the combined light beam from the beam mixer, wherein the combined light beam is emitted from a tip of the ablation catheter to simultaneously perform ablation therapy and to generate photoacoustic signals in the target tissue;
an ultrasound transducer for detecting the generated photoacoustic signals; and
a processor in communication with the ultrasound transducer for processing the photoacoustic signals to create photoacoustic images of the tip of the ablation catheter to allow tracking of the tip in real time simultaneous to ablation therapy.

16. The system of claim 15, wherein a wavelength of light from the pulsed laser is different than a wavelength of light from the continuous wave laser.

17. The system of claim 15, wherein the beam mixer includes a dichroic mirror, a hot mirror or a cold mirror.

18. The system of claim 15, wherein the ultrasound transducer is configured to transmit ultrasound signals to the target tissue for generating ultrasound images of the target tissue.

19. The system of claim 18, further comprising a display in communication with the processor for displaying the photoacoustic images, the ultrasound images, or both.

20. A system for monitoring an ablation procedure in a target tissue, the system comprising:
a pulsed laser for delivering light pulses to the target tissue to generate photoacoustic signals;
a continuous wave laser for delivering continuous wave light energy to the target tissue for ablation therapy;
a beam mixer for receiving light from the pulsed laser and the continuous wave laser to create a combined light beam;
an ablation catheter including a single optical fiber which receives the combined light beam from the beam mixer, wherein the combined light beam is emitted from a tip of the ablation catheter to simultaneously perform ablation therapy and to generate photoacoustic signals in the target tissue;
an ultrasound transducer for detecting the generated photoacoustic signals; and
a processor in communication with the ultrasound transducer for processing the photoacoustic signals to monitor a temperature at the tip of the ablation catheter in real time simultaneous to ablation therapy.
